(19) United States
(12) Patent Application Publication
(10) Pub. No.: US 2015/0265344 A1
(43) Pub. Date: Sep. 24, 2015

(54) CATHETER WITH INTEGRATED TRANSSEPTAL PUNCTURE NEEDLE

(71) Applicants: Mehmet Kemal AKTAS, Penfield, NY (US); Spencer Z. ROSERO, Pittsford, NY (US); David HUANG, Pittsford, NY (US)

(72) Inventors: Mehmet Kemal AKTAS, Penfield, NY (US); Spencer Z. ROSERO, Pittsford, NY (US); David HUANG, Pittsford, NY (US)

(73) Assignee: UNIVERSITY OF ROCHESTER, Rochester, NY (US)

(21) Appl. No.: 14/731,355

(22) Filed: Jun. 4, 2015

Related U.S. Application Data
(63) Continuation of application No. PCT/US2013/073438, filed on Dec. 5, 2013.
(60) Provisional application No. 61/733,676, filed on Dec. 5, 2012.

(51) Int. Cl. A61B 18/14

(52) U.S. Cl. CPC A61B 18/1492; A61B 2018/00196

(57) ABSTRACT

A cardiac treatment system of the subject technology provides access across an interatrial septum of a patient. Such a treatment system can include a catheter having an ablation treatment device on an outer surface of a distal region thereof. A needle is provided at least partially within a lumen of the catheter. A control unit is operatively connected to the ablation treatment device for ablation of a portion of a septum by RF ablation. A distal end of a catheter is provided to the septum (e.g., at the fossa ovalis) of the patient while a distal tip of the needle is within the lumen and proximal to the distal end of the catheter. The distal tip of the needle punctures and crosses the septum, and a portion of the septum is ablated with an ablation treatment device aligned with the septum by guidance from the needle.

### RELATED APPLICATIONS

The present application is a continuation of International Patent Application No. PCT/US2013/073438, filed Dec. 5, 2013, entitled CATHETER WITH INTEGRATED TRANSSEPTAL PUNCTURE NEEDLE, which claims the benefit of U.S. Provisional Application No. 61/733,676, filed Dec. 5, 2012, each of the foregoing applications being incorporated by reference in its entirety.

### FIELD

The subject technology is directed to a transseptal needle and more particularly to such a needle that is integrated into an ablation, perfusion, pressure, or mapping catheter or the like.

### BACKGROUND

The human heart includes a right ventricle, a right atrium, left ventricle, and left atrium. The right atrium is in fluid communication with the superior vena cava and the inferior vena cava. The tricuspid valve separates the right atrium from the right ventricle. The right atrium is separated from the left atrium by a septum that includes a thin membrane known as the fossa ovalis.

Diagnostic and therapeutic procedures have been developed in which a catheter is transluminally advanced within a guide sheath or over a guidewire into various chambers and across valves of the heart. Access to the left atrium can be achieved through the femoral or left subclavian vein into the right atrium, and subsequent penetration of the interatrial septum, the fossa ovalis, to gain entry to the left atrium. This procedure is commonly referred to as transseptal catheterization. The objectives of left atrial access can be diagnostic or therapeutic.

Conventional technologies for performing a transseptal puncture to gain access into the left atrium (LA) utilize separate stand-alone needle systems that require additional venous access. These stand-alone needle systems require exchanges in and out of the vascular system, which requires time and exposes the patient to risks such as vascular injury and introduction of air into the circulatory system.

### SUMMARY

A retractable transseptal needle can be integrated into an ablation, perfusion, pressure, and mapping catheter, allowing a user to perform a transseptal puncture and left atrial access without having to add additional sheaths and without having to change out for a stand-alone transseptal needle. The catheter has an integrated deployable-retractable needle, but can also be fixed, and can also include a transseptal needle and/or septal fixation device that is integrated into an ablation catheter that delivers radio-frequency (RF) energy or another treatment or diagnostic method, including but not limited to cryogenic/cold, microwave or ultrasound waves, to facilitate/perform the transseptal puncture across a septum dividing the left and right atrium, or a tissue/and/or prosthetic material barrier between two chambers/spaces.

The deployable-retractable transseptal needle system can be integrated into other catheters that require transseptal access into the systemic circulation for purposes such as left atrial appendage occlusion devices, mitral valvuloplasty, and atrial septal defect and patent foramen ovale closures. When integrated with a radio frequency (“RF”) ablation catheter, this deployable-retractable needle system can be used during ablation of mid-myocardial segments such as in ablation of ventricular tachyarrhythmias or for purposes of relieving outflow tract obstruction in patients with hypertrophic obstructive cardiomyopathy. This system can also be used in the delivery of therapeutic devices such as left ventricular pacing leads. The catheter has an inner lumen that can be insulated, through which the needle/puncture needle cable is inserted through the length of the ablation/and or hemodynamic catheter. The needle itself can or cannot have a lumen through which fluids including contrast dye can be irrigated to facilitate tissue staining. The forward (extension), backward (retraction), and torque (turning to deploy or bend) aspects of the needle or puncture device can be controlled from the proximal handle using an adapted controller or separate sliding and/or rotary, and/or torque based mechanism for extending, retracting, and turning of the needle/catheter system. The needle can be straight, coaxial, or made of multiple components, although a preferred embodiment has a single needle with a proximal cable welded to provide maneuverability.

The subject technology in at least some embodiments includes a proximal handle control mechanism that provides for the retraction of a relatively large length of the needle into the handle so as to decrease any change in the flexibility/range of motion of the distal working end of the catheter. This mechanism will likely require a pullback into a safe/storage mode of about 4-6 inches from the distal end adjusted for any particular catheter configuration.

The catheter-integrated transseptal system described herein can be used as part of sheath system with a long tapered distal portion that can be used to advance across the punctured septum. Advancement of the sheath across the septum can be necessary if the user wishes to maintain access to the chamber while the catheter is being replaced.

Having a catheter-integrated deployable-retractable transseptal catheter system will decrease the number of sheaths introduced into the vascular system and will avoid the need for sheath and catheter exchanges. The catheter-integrated transseptal needle system can also allow for manipulation and deflection of the catheter itself to facilitate transseptal access. In addition, having the needle integrated into an RF ablation catheter can allow for RF energy facilitated puncture across the fossa ovalis, obviating the need for specialized RF needles and special generators. Currently, access into the left atrium requires several steps, including use of a stand-alone needle used to perform the transseptal puncture. Once access into the left atrium is obtained with this needle, the dilator portion of a long sheath is advanced into the left atrium, and through the inner lumen of the dilator a wire is advanced into the left atrium, and finally, the dilator and long sheath are together advanced into the left atrium. Once the sheath is in the left atrium, the dilator is removed, and the ablation catheter is finally advanced into the left atrium. The catheter-integrated, deployable-retractable transseptal catheter will facilitate a transseptal puncture, and once access into the left atrium is established, the needle can be retracted, and the ablation catheter can be simply advanced into the left atrium. This will not only save crucial time and decrease the number of steps, but will also minimize the need for extended periods of fluoroscopy and the need for sheath and catheter change outs.

A method, according to some embodiments, can include: providing a distal end of a catheter to a septum of a heart of the patient while a distal tip of the needle is within a lumen and proximal to the distal end of the catheter; advancing the distal tip of a needle from the lumen of the catheter to puncture and cross the septum; advancing a distal region of the catheter over the needle to span the septum; and ablating a portion of the septum with an ablation treatment device on an outer surface of the distal region.

Providing the distal end of the catheter to the septum of a heart of the patient can include advancing the catheter to a right atrium of the heart. Providing the distal end of the catheter to the septum of a heart of the patient can include advancing the catheter while the distal tip of the needle is within the lumen at least 4 inches from the distal end of the catheter. The method can include irrigating at least a portion of the heart with a fluid delivered from the lumen of the catheter while the needle is at least partially disposed in the lumen. Ablating the portion of the septum can include sending an RF signal to electrodes of the ablation treatment device.

A treatment system, according to some embodiments, can include: a catheter having a proximal end, a distal end, a lumen extending from the proximal end to the distal end, and a distal region comprising an ablation treatment device on an outer surface of the distal region; a needle disposed at least partially within the lumen and having a distal tip advanceable relative to the catheter; a control unit disposed at the proximal end of the catheter and being operatively connected to the ablation treatment device for ablation of a portion of a septum.

The needle can include a plurality of annular stages having a collapsed configuration and an extended configuration, the annular stages having a greater degree of overlap in the collapsed configuration than in the extended configuration. A distal annular stage can include a stopper on an outer surface of the distal annular stage and in contact with an inner surface of a proximal annular stage while in the extended configuration. The proximal annular stage can include an inner collar extending radially inward from the inner surface of the proximal annular stage, the inner collar having an inner cross-sectional dimension less than an outer cross-sectional dimension of the stopper. The distal annular stage can include an outer collar extending radially outward from the outer surface of the distal annular stage, the outer collar having an outer cross-sectional dimension greater than an inner cross-sectional dimension of the proximal annular stage. The distal tip can be disposed on a distalmost one of the annular stages. A distalmost one of the annular stages can be in fluid communication with a fluid controller.

The ablation treatment device can include an anode electrode and a cathode electrode. The controller can be configured to transmit RF signals to at least one of the anode electrode and the cathode electrode. The needle can be electrically insulated from the catheter. The ablation treatment device can include a passage for directing a flow of cryogenic fluid to the distal region.

A method, according to some embodiments, can include: providing a distal end of a catheter to a septum of a heart of the patient; advancing a distal tip of a needle from a lumen of the catheter to puncture and cross the septum by transitioning a plurality of annular stages of the needle from a collapsed configuration to an extended configuration, the annular stages having a greater degree of overlap in the collapsed configuration than in the extended configuration; advancing a distal region of the catheter over the needle to span the septum; and ablating a portion of the septum with an ablation treatment device on an outer surface of the distal region.

Advancing the distal tip can include advancing a distal annular stage of the needle, carrying the distal tip, relative to a proximal annular stage of the needle. Advancing the distal tip can include increasing a fluid pressure against an inner surface of the distal tip to an extended configuration pressure; sensing, by a processor, a return force applied by the septum against the distal tip as the distal tip contacts the septum; sensing, by a processor, when the return force is reduced as the distal tip extends beyond the septum. Sensing the return force can include determining that the fluid pressure exceeds the extended configuration pressure. The method can include sensing, by a processor, when the return force is reduced includes determining that the fluid pressure has returned to the extended configuration pressure. Providing the distal end of the catheter to the septum of a heart of the patient can include advancing the catheter to a right atrium of the heart.

The method can include irrigating at least a portion of the heart with a fluid delivered from the lumen of the catheter while the needle is at least partially disposed in the lumen.

Additional features and advantages of the subject technology will be set forth in the description below, and in part will be apparent from the description, or can be learned by practice of the subject technology. The advantages of the subject technology will be realized and attained by the structure particularly pointed out in the written description and claims hereof as well as the appended drawings.

It is to be understood that both the foregoing general description and the following detailed description are exemplary and explanatory and are intended to provide further explanation of the subject technology as claimed.

### BRIEF DESCRIPTION OF THE DRAWINGS

The accompanying drawings, which are included to provide further understanding of the subject technology and are incorporated in and constitute a part of this specification, illustrate aspects of the subject technology and together with the description serve to explain the principles of the subject technology.

FIG. 1A shows an elevation view of a treatment system having ablation electrodes, according to some embodiments of the subject technology.

FIG. 1B shows cross-sectional view of a treatment system having ablation electrodes, according to some embodiments of the subject technology.

FIG. 2 shows cross-sectional view of a treatment system having cryoablation passages, according to some embodiments of the subject technology.

FIG. 3A shows cross-sectional view of a treatment system having needle control mechanisms, according to some embodiments of the subject technology.

FIG. 3B shows cross-sectional view of a treatment system having needle control mechanisms, according to some embodiments of the subject technology.

FIG. 4A shows cross-sectional view of a treatment system having needle control mechanisms, according to some embodiments of the subject technology.

FIG. 4B shows cross-sectional view of a treatment system having needle control mechanisms, according to some embodiments of the subject technology.

FIG. 5A shows a partially cutaway perspective view of a multistage needle in a collapsed position, according to some embodiments of the subject technology.

FIG. 5B shows a cross-sectional elevational view of the multistage needle of FIG. 5A in a collapsed position, according to some embodiments of the subject technology.

FIG. 5C shows an enlarged cross-sectional elevational view of a portion of the multistage needle of FIG. 5B, according to some embodiments of the subject technology.

FIG. 6A shows a partially cutaway perspective view of a multistage needle in an extended position, according to some embodiments of the subject technology.

FIG. 6B shows a cross-sectional elevational view of the multistage needle of FIG. 6A in an extended position, according to some embodiments of the subject technology.

FIG. 6C shows an enlarged cross-sectional elevational view of a portion of the multistage needle of FIG. 6B, according to some embodiments of the subject technology.

FIG. 7A shows a cross-sectional elevational view of a multistage needle in a collapsed position, according to some embodiments of the subject technology.

FIG. 7B shows an enlarged cross-sectional elevational view of a portion of the multistage needle of FIG. 7A, according to some embodiments of the subject technology.

FIG. 8A shows a schematic view of a catheter positioned within the right atrium, according to some embodiments of the subject technology.

FIG. 8B shows a schematic view of a catheter positioned within the right atrium, according to some embodiments of the subject technology.

FIG. 9A shows a schematic view of a needle emerging from the catheter within the right atrium, penetrating the interatrial septum (e.g., at the fossa ovalis), and entering the left atrium through a puncture hole, according to some embodiments of the subject technology.

FIG. 9B shows a schematic view of the catheter penetrating the interatrial septum (e.g., at the fossa ovalis), and entering the left atrium through the puncture hole, with a treatment device aligned with the interatrial septum, according to some embodiments of the subject technology.

FIG. 9C shows a schematic view of the treatment device ablating a portion of the interatrial septum (e.g., at the fossa ovalis), according to some embodiments of the subject technology.

FIG. 10A shows a schematic view of a needle emerging from a sheath within the right atrium, penetrating an interatrial septum (e.g., at the fossa ovalis), and entering the left atrium through a puncture hole, according to some embodiments of the subject technology.

FIG. 10B shows a schematic view of the sheath penetrating the interatrial septum (e.g., at the fossa ovalis), and entering the left atrium through the puncture hole, according to some embodiments of the subject technology.

FIG. 10C shows a schematic view of the sheath having been retracted to uncover a catheter extending through the puncture hole, with a treatment device of the catheter aligned with the interatrial septum, according to some embodiments of the subject technology.

FIG. 11A shows a schematic view of a catheter positioned within the right atrium, according to some embodiments of the subject technology.

FIG. 11B shows a schematic view of a multistage needle emerging from the catheter within the right atrium and contacting an interatrial septum (e.g., at the fossa ovalis), according to some embodiments of the subject technology.

FIG. 11C shows a schematic view of the multistage needle emerging from the catheter within the right atrium, penetrating the interatrial septum (e.g., at the fossa ovalis), and entering the left atrium through a puncture hole, according to some embodiments of the subject technology.

### DETAILED DESCRIPTION

In the following detailed description, numerous specific details are set forth to provide a full understanding of the subject technology. It will be apparent, however, to one ordinarily skilled in the art that the subject technology can be practiced without some of these specific details. In other instances, well-known structures and techniques have not been shown in detail so as not to obscure the subject technology.

A phrase such as “an aspect” does not imply that such aspect is essential to the subject technology or that such aspect applies to all configurations of the subject technology. A disclosure relating to an aspect can apply to all configurations, or one or more configurations. An aspect can provide one or more examples of the disclosure. A phrase such as “an aspect” can refer to one or more aspects and vice versa. A phrase such as “an embodiment” does not imply that such embodiment is essential to the subject technology or that such embodiment applies to all configurations of the subject technology. A disclosure relating to an embodiment can apply to all embodiments, or one or more embodiments. An embodiment can provide one or more examples of the disclosure. A phrase such “an embodiment” can refer to one or more embodiments and vice versa. A phrase such as “a configuration” does not imply that such configuration is essential to the subject technology or that such configuration applies to all configurations of the subject technology. A disclosure relating to a configuration can apply to all configurations, or one or more configurations. A configuration can provide one or more examples of the disclosure. A phrase such as “a configuration” can refer to one or more configurations and vice versa.

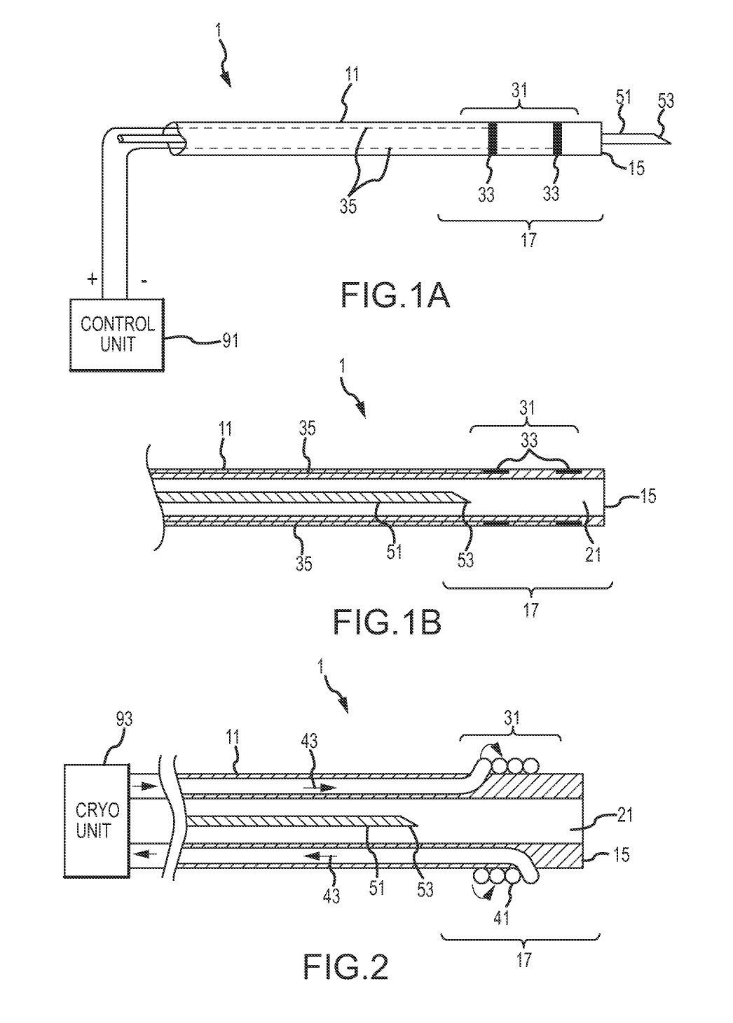

According to some embodiments, a treatment system 1 provides access and treatment capabilities for treating a patient. As shown in FIG. 1A, the treatment system 1 includes a catheter 11 that extends along a longitudinal axis to a distal end 15. The treatment system 1 can further include a treatment device 31 located at a distal region 17 of the catheter 11. The treatment device 31 can be located on a radially outward surface of the catheter 11 at the distal region 17. The treatment device 31 can be located at a distalmost end 15 of the catheter 11.

The treatment device 31 can be configured to treat tissue of a patient in the vicinity of the treatment device 31. The treatment device 31 can be an ablation device configured to ablate tissue in the vicinity of the treatment device 31. For example, as shown in FIGS. 1A-B, the treatment device 31 can include one or more electrodes 33. The electrodes 33 can extend entirely or partially circumferentially about an outer surface of the catheter 11 (e.g., at the distal region 17). The electrodes of the treatment device 31 can form a monopolar electrode system, a bipolar electrode system, or a multi-polar electrode system.

According to some embodiments, at least one of the electrodes 33 is a cathode electrode. According to some embodiments, at least one of the electrodes 33 is an anode electrode. For example, at least two of the electrodes 33 can be configured to be oppositely charged. The longitudinal distance between electrodes can be configured to match or exceed a span of a septum to be ablated.

Each or a plurality of electrodes 33 can be connected to one or more leads 35. The leads 35 can extend from one or more electrodes 33 to an ablation control unit 91. The ablation control unit 91 is configured to operatively control, activate, deactivate, and/or monitor the electrodes 33. The ablation control unit 91 can include a power supply and programmable logic for operating the electrodes 33 according to a program or according to user input.

The ablation control unit 91 provides RF energy to one or more electrodes 33 to facilitate a transseptal procedure. In some instances, the energy is returned through one of the electrodes 33 of the treatment system 31, which can also be coupled to the electrode control unit.

According to some embodiments, the leads 35 connecting electrodes 33 of the treatment system 31 to the ablation control unit 91 can be electrically isolated from other components of the treatment system 1. For example, as shown in FIG. 1B, the leads 35 can reside within layers of the catheter 11, such that the leads 35 are electrically isolated from the lumen 21 and objects within the lumen 21, such as the needle 51. Materials for the catheter 11 can be electrically insulative, and insulative coating can be applied to an inner surface of the catheter 11, or additional structure can be provided between the needle 51 and the leads 35 for electrically insulating the needle 51 from the leads 35. By further example, the leads 35 can be electrically isolated from components radially outward from the catheter 11 (e.g., contacting a radially outward surface of the catheter 11).

As shown in FIGS. 1A-B, the treatment system 1 includes a needle 51. The needle 51 can be located entirely or at least partially within the catheter 11. For example, as shown in FIG. 1A, a distal tip 53 of the needle 51 can extend distally of the distal end 15 of the catheter 11. By further example, as shown in FIG. 1B, the distal tip 53 of the needle 51 can be located proximally of the distal end 15 of the catheter 11. The needle 51 can have any configuration, including tapered, conical, frustoconical, pointed, sharpened, wedged, serrated, knifelike, helical, or coiled. The needle 51 can provide a lumen along a longitudinal axis thereof.

As shown in FIG. 1B, the catheter 11 of the treatment system 1 includes a lumen 21. The needle 51 can be entirely or at least partially housed within the lumen 21 of the catheter 11. The lumen 21 can provide an opening or port at the distal end 15 of the catheter 11.

According to some embodiments, the catheter 11 and/or the needle 51 can provide an aperture allowing a corresponding lumen to be in fluid communication with an exterior of the corresponding catheter 11 or needle 51. Irrigation and/or aspiration can be provided via the lumens. For example, a saline solution can be injected via the catheter 11 and/or the needle 51. An aperture can be controllably opened or closed to provide or prevent fluid communication there through. An aperture can be present at a distal end or through a side wall of a corresponding structure.

According to some embodiments, as shown in FIG. 2, the treatment device 31 can include a cryoablation device. The treatment device 31 can include passages 41 on a radially outer surface of the catheter 11 and or at a distal end 15 of the catheter 11. The passages 41 provide a supply pathway for a flow 43 of a cryogenic fluid. Cryogenic cooling can occur adjacent the passages 41 at the distal region 17 of the catheter 11 as a result of a liquid-to-gas phase change that takes place in the cryogenic fluid. A cryogenic fluid such as liquid nitrous oxide is supplied by a cryogenic control unit 93. The transformation is accompanied by rapid and extreme cooling, as temperatures as low as −60° C. can be attained. Other temperatures are contemplated for ablating tissue. The cryogenic fluid is conducted away from the distal region 17 through a return pathway of the flow 43. The cryogenic fluid can be vented to the atmosphere or recycled. The passages 41 can be formed from any suitable material having a high coefficient of thermal conductivity, such as silver, gold, and platinum. As shown in FIG. 2, the passages 41 can form a coil or reservoir at the distal region 17 of the catheter 11. The longitudinal distance of the treatment device 31 with passages 41 can be configured to match or exceed a span of a septum to be ablated.

According to some embodiments, the treatment device 31 can include other ablation devices, such as ultrasound treatment devices. The treatment device 31 can be used in conjunction with other devices located outside of the patient. For example, the treatment device 31 can independently or collaboratively provide high intensity focused ultrasound ablation of a septum. Other mechanisms and methods for ablating a septum or other tissue of the patient are contemplated for use in conjunction with the treatment system 1 of the subject technology.

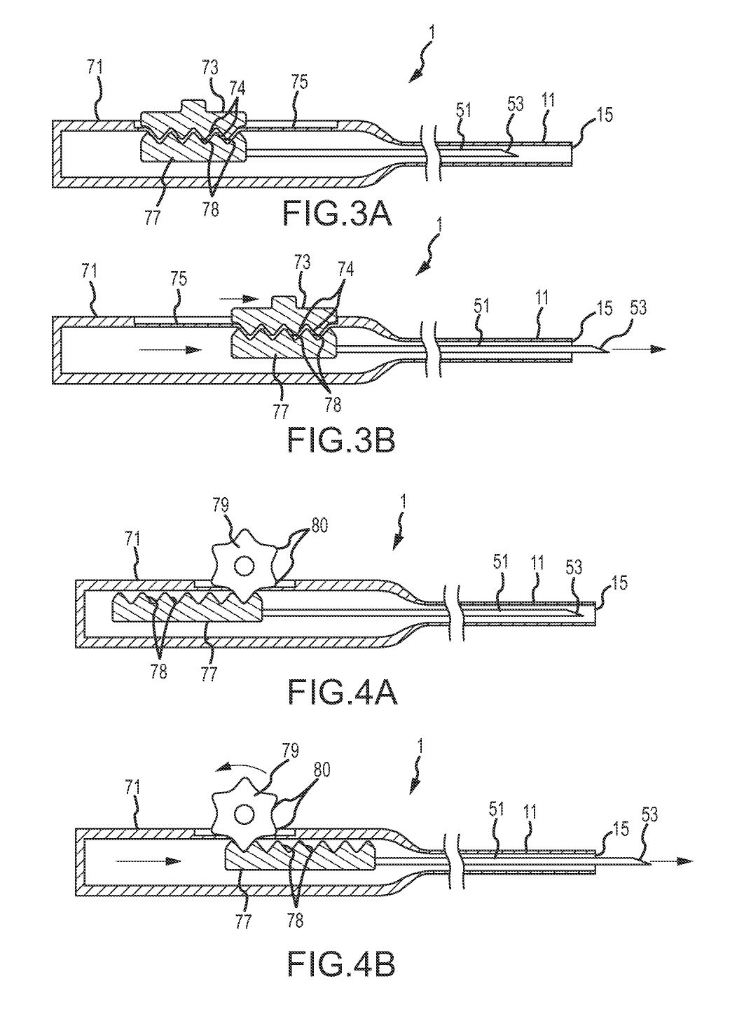

According to some embodiments, as shown in FIGS. 3A-B, advancement and retraction of the needle 51 can be effectuated by a user operating a handle 73 of a needle control device 71. As shown, the handle 73 can include teeth 74 for engaging complementary shaped teeth 78 of a needle interface member 77. The needle interface number 77 can be fixedly attached to a proximal end of the needle 51, such that longitudinal movement of the handle 73 and the needle interface member 77 results in corresponding longitudinal movement of the needle 51 and the distal tip 53 of the needle 51. The handle 73 can be controllably moved along a track 75.

According to some embodiments, as shown in FIGS. 4A-B, advancement and retraction of the needle 51 can be effectuated by a user operating a wheel 79 of the needle control device 71. As shown, the wheel 79 can include teeth 80 for engaging the teeth 78 of the needle interface member 77. The wheel 79 and the needle interface member 77 can be placed in a rack-and-pinion arrangement, such that rotational movement of the wheel 79 results in longitudinal movement of the needle interface member 77, the needle 51, and the distal tip 53 of the needle 51. Any number or arrangement of gears can be provided to transfer forces from the wheel 79 to the needle interface member 77.

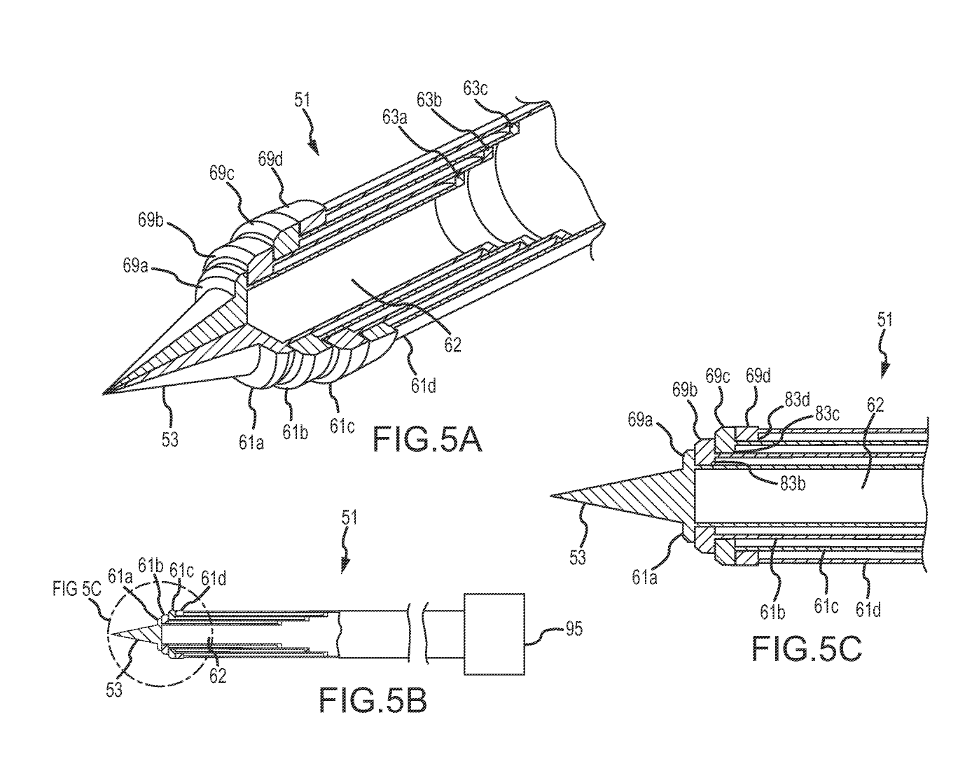

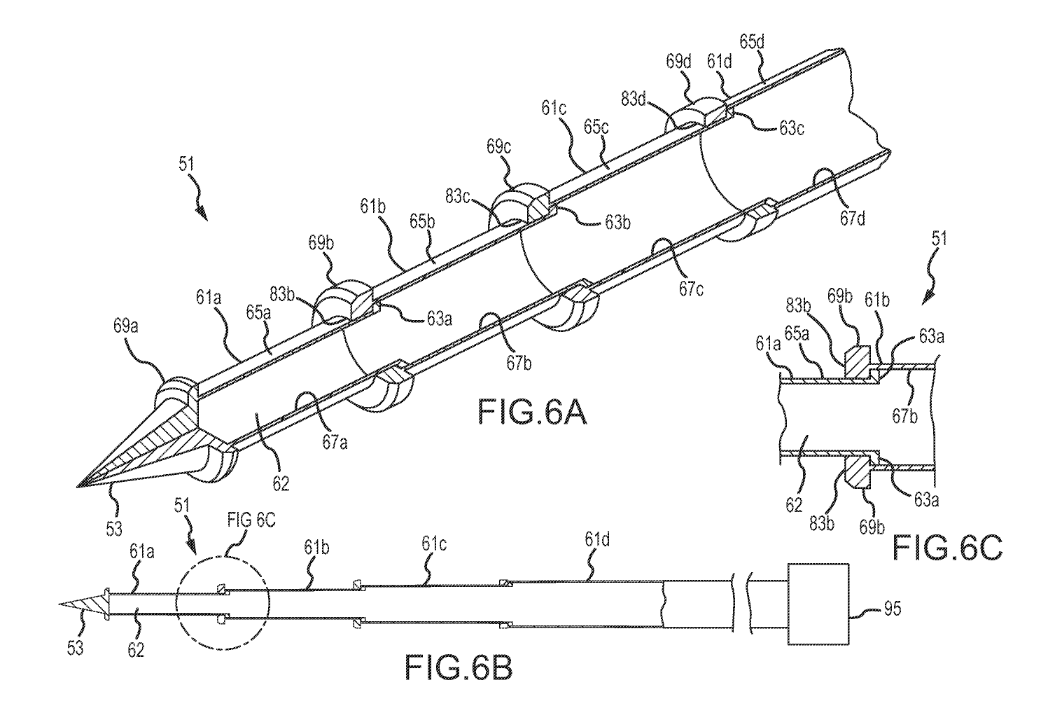

According to some embodiments, FIGS. 5A-C shows a multistage needle 51 of in a collapsed position. FIGS. 6A-C illustrate the multistage needle 51 of FIGS. 5A-C in an extended position. The multistage needle 51 has two or more annular stages 61. For example, as shown in FIGS. 5A-6C, the multistage needle 51 can include a proximalmost annular stage 61d, intermediate annular stages 61b and 61c, and distalmost annular stage 61a. The multistage needle 51 can include only two annular stages. The multistage needle 51 can include 2, 3, 4, 5, 6, 7, 8, or more annular stages. The distal tip 53 can be located on the distalmost annular stage 61a. In operation, the annular stages can extend one at a time until the multistage needle 51 is fully extended.

According to some embodiments, each annular stage 61 defines an inner surface 67 and an outer surface 65. The inner surface 67 and/or the outer surface 65 can extend as a hollow cylinder along or about a longitudinal axis of the needle 51. The annular stages 61 can be concentric or coaxial. According to some embodiments, the inner collars 83 and/or the outer surfaces 65 provide bearings which can maintain adjacent annular stages 61 in concentric spaced relation with each other. According to some embodiments, the outer collars 69 and/or the inner surfaces 67 provide bearings which can maintain adjacent annular stages 61 in concentric spaced relation with each other.

According to some embodiments, a channel 62 is defined through a plurality of annular stages 61. The proximalmost annular stage 61d includes an open distal end and a proximal end in fluid communication with a hydraulic control unit 95. The hydraulic control unit 95 is configured to controllably provide hydraulic fluid to the channel 62 for controllably extending and collapsing the multistage needle 51. The intermediate annular stages 61b,c each include an open proximal end and an open distal end. The distalmost annular stage 61a includes an open proximal end and a closed distal end.

According to some embodiments, a stopper 63 is located about a proximal end portion of one or more of the annular stages 61 (e.g., annular stages 61a,b,c). Each stopper 63 extends entirely or partially circumferentially about and radially outward from an outer surface 65 of the respective annular stage 61. The annular stages 61 support bearings (for example comprising wear bands) and/or seals disposed between the annular stages to prevent hydraulic fluid from flowing out of the channel 62. As shown in FIG. 6C, the bearings and/or seals can be located on an outer surface of the stoppers 63 to provide a sealed engagement between the stoppers 63 and a corresponding inner surface 67 of an adjacent annular stage 61. The sealed engagement can be maintained for all relative positions (i.e., degrees of extension) of two adjacent annular stages 61. For example, the sealed engagement can be maintained in an extended configuration and a collapsed configuration.

According to some embodiments, an outer collar 69 is located at a distal end portion of one or more of the annular stages 61 (e.g., annular stages 61a,b,c,d). Each outer collar 69 extends radially outward from an outer surface 65 of an annular stage 61. In the collapsed configuration, as shown in FIG. 5C, the outer collar 69a of a distal annular stage 61a abuts a more proximally positioned inner collar 83b and/or outer collar 69b of a proximal annular stage 61b to prevent further proximally directed movement of the distal annular stage 61a relative to the proximal annular stage 61b. The inner collar 83b of the proximal annular stage 61b can have an inner cross-sectional dimension less than an outer cross-sectional dimension of the stopper 83a.

According to some embodiments, an inner collar 83 is located at a distal end portion of one or more of the annular stages 61 (e.g., annular stages 61b,c,d). The inner collars 83 may be located at distalmost ends or located proximally of the distalmost ends of the corresponding annular stage. Each inner collar 83 extends radially inward from an inner surface 67 of an annular stage 61. In the expanded configuration, as shown in FIG. 6C, the stopper 63a of a distal annular stage 61a abuts a more approximately positioned inner collar 83b of a proximal annular stage 61b to prevent further distally directed movement of the distal annular stage 61a relative to the proximal annular stage 61b. The outer collar 69b of the distal annular stage 61b can have an outer cross-sectional dimension greater than an inner cross-sectional dimension of the proximal annular stage 61a (e.g., at the inner collar 83a). Such structures and configurations can apply to each axially adjacent pairing of annular stages 61.

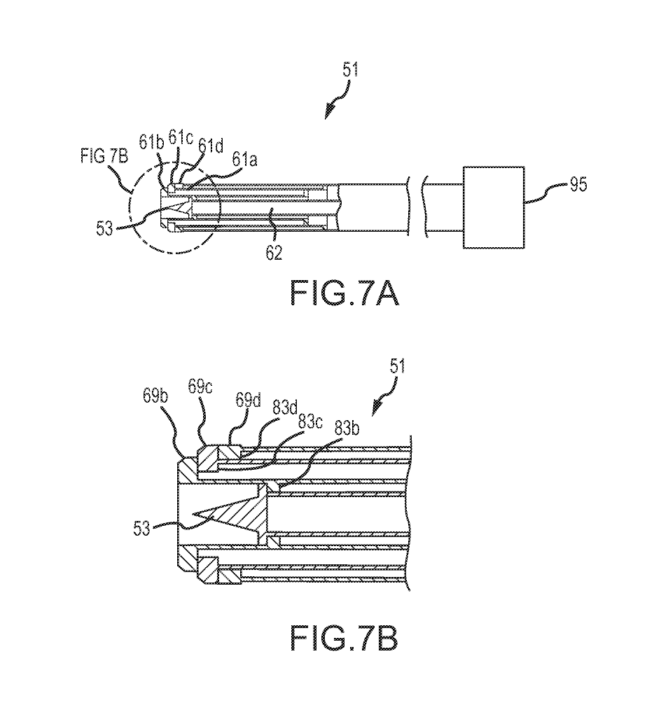

According to some embodiments, as shown in FIG. 7A-7B, at least one inner collar 83 can be positioned proximally of the distal end of the corresponding annular stage 61. As shown in FIG. 7B, the inner collar 83b of an intermediate annular stage 61b is positioned to allow the distalmost annular stage 61a to be fully housed and contained within an interior of the intermediate annular stage 61b, such that no portion of the distal tip 53 extends beyond the distal and of the intermediate annular stage 61b. For example, the distance between the distal end of the annular stage 61b and the inner collar 83b is at least as long as the distal tip 53. By further example, the distance between the distal end of the annular stage 61b and the inner collar 83b is at least as long as the entire length of the distalmost annular stage 61a, such that no portion of the distal tip 53 of the distalmost annular stage 61a extends distally beyond the distal end of the proximal annular stage 61b while in the collapsed configuration. As such, the needle 51 provides a flatter, smoother, and less traumatic surface at the distal end thereof while in the collapsed configuration. Other inner collars 83 can be positioned proximally of the distalmost end of a corresponding annular stage 61, such that the distalmost faces of a plurality of annular stages 61 are axially aligned to provide an aggregate flat or atraumatic distalmost surface. For example, inner collars 83 can be positioned a distance from a distalmost end of its corresponding annular stage 61 substantially equal to the width of an outer collar 69 of an adjacent annular stage 61.

A raised hydraulic pressure provided by a hydraulic fluid in the channel 62 can apply a distally directed force to the closed distal end of the distalmost annular stage 61a, to cause the distalmost annular stage 61a to extend relative to at least one other annular stage 61. A raised hydraulic pressure can be a pressure within the channel 62 that is greater than a pressure outside of the multistage needle 51. Likewise, a reduced hydraulic pressure provided in the channel 62 can apply a proximally directed force to the closed distal end of the distalmost annular stage 61a, to cause the distalmost annular stage 61a to collapse and retract relative to at least one other annular stage 61. A reduced hydraulic pressure can be a pressure within the channel 62 that is less than a pressure outside of the multistage needle 51. The multistage needle 51 can be provided with a pressure sensor or a force sensor to measure a pressure or force applied to one or more of the stages 61. For example, a pressure sensor can be located within any one or more of the stages 61 or at the hydraulic control unit 95 in fluid communication with the channel 62 to determine pressure conditions within the channel 62. Parameters sensed or measured by sensors of the system can be communicated to and processed by the hydraulic control unit 95. For example, a pressure within the channel 62 can be recorded before the needle 51 contacts the septum, as the needle 51 initially contacts the septum, as the needle 51 punctures the septum, as the needle 51 traverses the septum, and after the needle has extended at least partially beyond the septum. Parameters can be measured and recorded for one or more of such phases of a procedure, as discussed further herein.

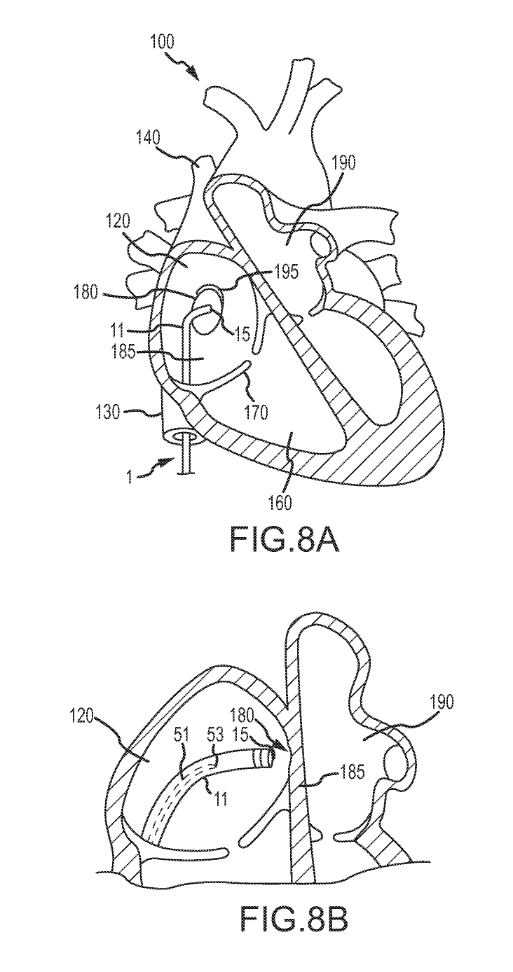

FIG. 8A shows a diagrammatic representation of a heart 100 that illustrates relevant anatomic structures within the interior chamber of a right atrium 120. The right atrium 120 receives blood from an inferior vena cava 130 and a superior vena cava 140. Blood from the right atrium 120 empties into a right ventricle 160 via a tricuspid valve 170. A fossa ovalis 180, a discrete portion of an interatrial septum 185 that lies between the right atrium 120 and the left atrium 190, is shown with its approximate position relative to the other structures. The fossa ovalis is located posterior and caudal to an aortic root, anterior to a free wall of the right atrium, superiorly and posteriorly to an ostium of the coronary sinus, and posterior of a tricuspid annulus and a right atrial appendage. In an average human heart, the fossa ovalis ranges from 2 to 10 mm in diameter and is bounded superiorly by a ridge known as a limbus 195.

In a transseptal procedure, the fossa ovalis 180 can be punctured to gain access to the left atrium 190, which lies on the other side of the fossa 180. Depending upon the desired application or procedure, the practitioner can puncture different areas of the fossa ovalis 180. For example, during mitral valve replacements or repairs, the practitioner can puncture at a posterior and superior area of the fossa ovalis 180. In contrast, for occlusion of the left atrial appendage, the practitioner can puncture a posterior and more central area of the fossa ovalis 180.

As shown in FIG. 8A, the catheter 11 of the system 1 is shown positioned within the right atrium 120 via the inferior vena cava 130 such that the distal end 15 of the catheter 11 is situated near the fossa ovalis 180. It is understood that alternative access paths can be utilized to place the catheter 11 into the right atrium 120. For example, alternatively, the catheter 11 could be inserted into the right atrium 120 via the superior vena cava 140. After positioning the distal end 15 of the catheter 11 within the right atrium 120 using standard percutaneous access methods, the medical practitioner can reposition the catheter 11 by rotating, retracting, or advancing the catheter 11.

According to some embodiments, as shown in FIGS. 8A-B, a catheter 11 can be positioned within the right atrium 120. Visualization techniques such as intracardiac echocardiography (ICE) or magnetic resonance imaging (MRI) can be used to confirm accurate positioning of the catheter 11. As the catheter 11 is brought to the right atrium and positioned near the interatrial septum 185 (e.g., at the fossa ovalis 180), the distal tip 53 of the needle 51 can be within the lumen 21 at a position proximal of the distal end 15 of the catheter 11. This arrangement provides a distal portion of the catheter 11 with a greater degree of flexibility. Preferably, the distal tip 53 of the needle 51 can be at least about 4 inches proximal of the distal end 15 of the catheter 11. The distal tip 53 of the needle 51 can be about 1 inch, about 2 inches, about 3 inches, about 4 inches, about 5 inches, about 6 inches, about 7 inches, about 8 inches, about 9 inches, or about 10 inches proximal of the distal end 15 of the catheter 11.

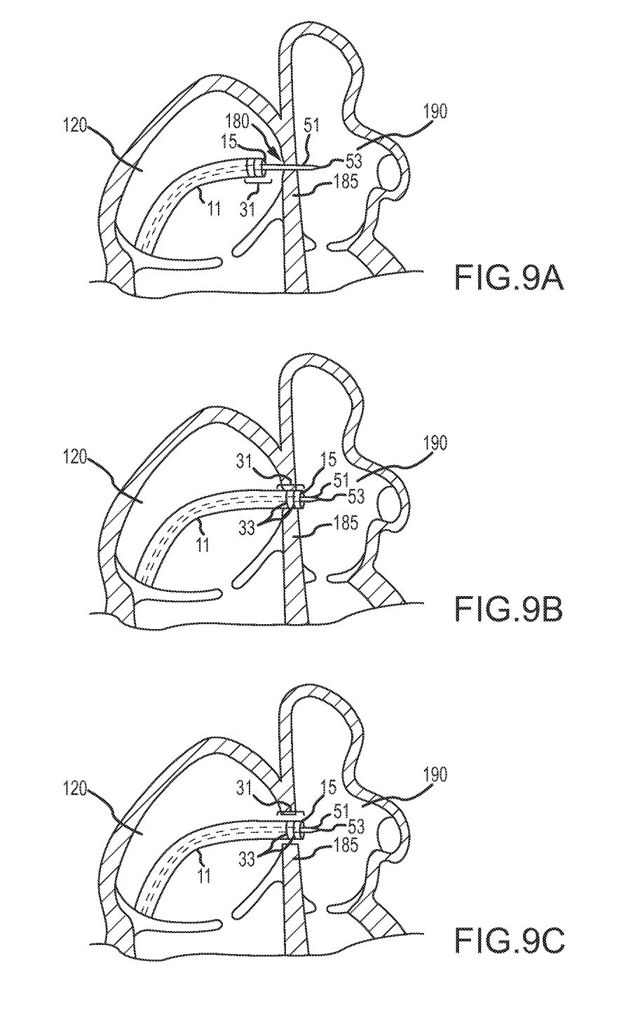

According to some embodiments, as shown in FIG. 9A, a needle 51 can emerge from the catheter 11 within the right atrium 120, penetrate the interatrial septum 185 (e.g., at the fossa ovalis 180), and enter the left atrium 190 through a puncture hole. As shown in FIG. 9B, the catheter 11 can follow a path defined by the needle 51 to penetrate the interatrial septum 185 (e.g., at the fossa ovalis 180) and enter the left atrium 190 through the puncture hole, with a treatment device 31 aligned with the interatrial septum 185. According to some embodiments, the needle 51 can be retracted before or after a treatment phase of the treatment device 31. According to some embodiments, irrigation can be provided via the lumen 21 before, during, or after a treatment phase of the treatment device 31. The irrigation can include injection of a fluid such as saline. The irrigation can be provided with the needle 51 in the lumen 21 or after the needle 51 is removed from the lumen 21. The irrigation can be provided via a lumen of the needle 51.

According to some embodiments, as shown in FIG. 9C, the treatment device 31 can ablate a portion of the interatrial septum 185 (e.g., at the fossa ovalis 180). According to some embodiments, to widen the puncture hole in the interatrial septum 185 by ablating the septal tissue, the ablation control unit 91 (shown in FIG. 1) can be activated to supply the electrodes 33 with enough RF energy (i.e., a sufficiently high potential) to ionize or break down the liquid contained in the septal tissue located adjacent to the electrodes 33. An ionizing arc between electrodes 33 is used to convert the solid septal tissue into a plasma state, thereby effectively vaporizing the septal tissue into particulate matter that is safely absorbed by the blood stream. Once the spark erosion process is initiated, a lower energy potential can be employed to maintain the ablation as the electrodes 33 can be moved against the tissue. While the electrodes 33 supply RF energy to the septal tissue, the catheter 11 can be moved in a forward, backward, reciprocating, linear, and/or rotational fashion, for example, to widen the puncture hole. According to some embodiments, other mechanisms for ablating septal tissue can be used, as disclosed herein, such as cryoablation and/or ultrasound techniques.

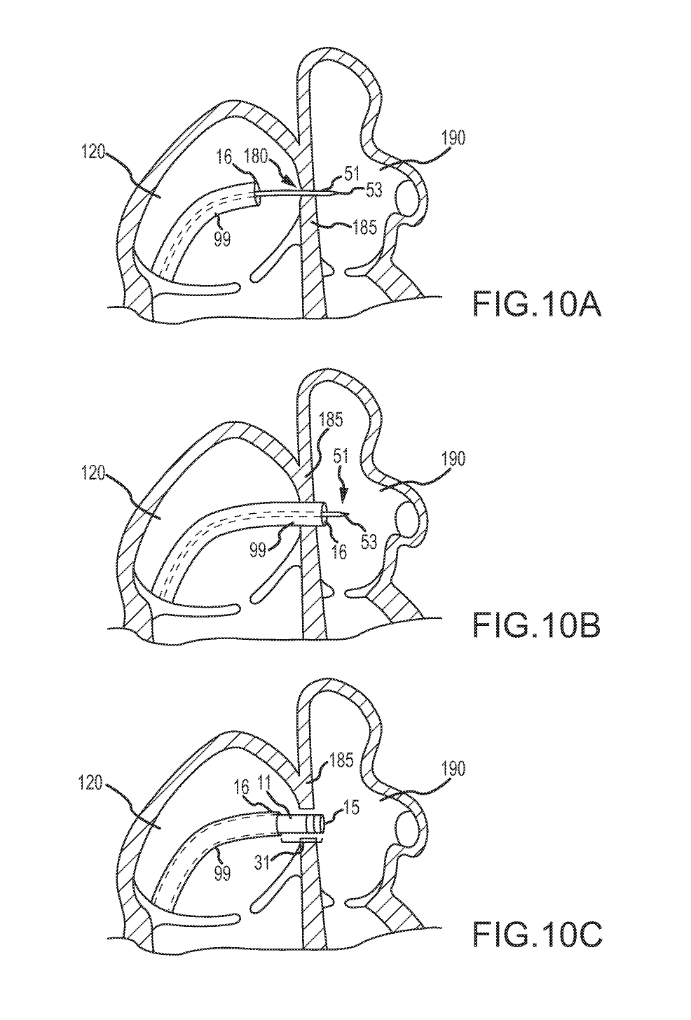

According to some embodiments, as shown in FIG. 10A, a needle 51 can emerge from a sheath 99 positioned within the right atrium 120, penetrate an interatrial septum 185 (e.g., at the fossa ovalis 180), and enter the left atrium 190 through a puncture hole.

As shown in FIG. 10B, the sheath 99 can follow a path defined by the needle 51 to penetrate the interatrial septum 185 (e.g., at the fossa ovalis 180) and enter the left atrium 190 through the puncture hole. After advancing the sheath 99, a catheter 11 can be advanced within a lumen of the sheath 99 while the sheath 99 spans the septum 185. The catheter 11 can be positioned such that the catheter 11 expands the septum 185. As shown in FIG. 10C, the sheath 99 can be refracted to uncover the catheter 11 extending through the puncture hole, and a treatment device 31 of the catheter 11 can be aligned with the interatrial septum 185.

With the treatment device 31 aligned with the interatrial septum 185, the treatment device 31 can ablate a portion of the interatrial septum 185, as disclosed herein with respect to FIG. 9C.

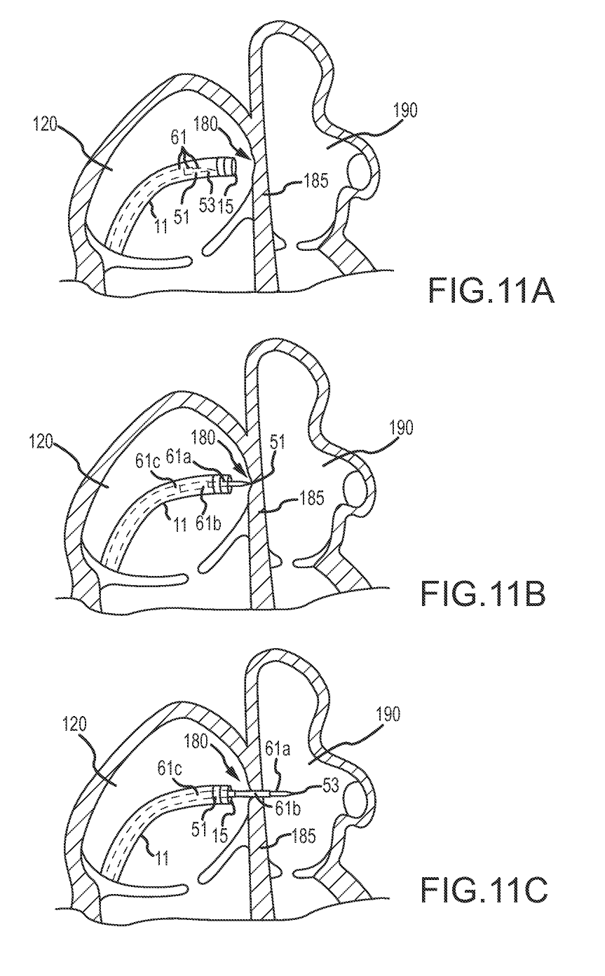

According to some embodiments, as shown in FIG. 11A, a catheter 11 can be positioned within the right atrium. As shown in FIG. 11B, a multistage needle 51 can emerge from the catheter 11 within the right atrium 120 and contact an interatrial septum 185 (e.g., at the fossa ovalis 180). According to some embodiments, the multistage needle 51 contacts the interatrial septum 185 with a distal tip 53.

According to some embodiments, the multistage needle 51 contacts the interatrial septum 185 with an atraumatic structure other than the distal tip 53. For example, as disclosed with respect to FIGS. 7A-B, the distal tip 53 can be refracted within an adjacent annular stage other than the distalmost stage. The adjacent annular stage other than the distalmost stage can provide an atraumatic surface from contacting and probing the interatrial septum 185. After the catheter 11 is properly positioned in the right atrium 120, the needle 51 can be advanced distally toward the interatrial septum 185. The atraumatic surface of a proximal stage 61b can be positioned against the fossa ovalis 180 at a desired puncture site and pushed against the fossa ovalis 180 until some tenting of the fossa ovalis results. In embodiments utilizing an imaging probe, the tenting can be observed by the imaging probe to correctly identify and confirm the puncture site. For example, the atraumatic surface of a proximal stage 61b can have a blunt distal tip that is unsuitable for penetration of the interatrial septum 185. The distal end of the proximal stage 61b, instead of the distal tip 53 of the distalmost stage 61a, can be positioned against the fossa ovalis 180 to position the needle 51 against the fossa ovalis 180 before the medical practitioner advances the distal tip 53 of the distalmost stage 61a through a puncture hole and into the left atrium 190 by actuation of the distalmost stage 61a. Actuation of the distal tip 53 of the distalmost stage 61a can be effected by a user and/or a hydraulic control unit 95, as disclosed herein.

As shown in FIG. 11C, the multistage needle 51 can emerge from the catheter 11 within the right atrium 120, penetrate the interatrial septum 185 (e.g., at the fossa ovalis), and enter the left atrium 190 through a puncture hole. The distal tip 53 of the multistage needle 51 can be advanced by advancing the entire length of the multistage needle 51 or by actuating a distalmost stage 61a of the multistage needle 51. Accordingly, the distal tip 53 can remain in the catheter 11 while the multistage needle 51 is in the collapsed configuration. Subsequently, the distal tip 53 can be advanced out of the catheter 11 by actuating the distalmost annular stage 61a of the multistage needle 51. The distal tip 53 can also be advanced out of the catheter 11 while the multistage needle 51 is in a collapsed configuration. Subsequently, the distal tip 53 can be advanced out of the catheter 11 by actuating the distalmost annular stage 61a of the multistage needle 51. Advancement and/or actuation of the multistage needle 51 can cause at least a portion of the multistage needle 51 to contact, puncture, or spend the interatrial septum 185.

According to some embodiments, a pressure within the channel 62 is measured after the needle 51 transitions from the collapsed configuration to the extended configuration. The initial pressure in the extended configuration can be recorded by the hydraulic control unit 95. As the needle 51 contacts, punctures, and advances across the interatrial septum 185, the pressure within the channel 62 can increase as the needle 51 and the septum 185 apply forces upon each other. For example, as a return force is applied by the septum 185 upon the needle 51, the annular stage is 61 can tend to collapse. The pressure in the channel 62 can be maintained to prevent such collapse, however the pressure in the channel 62 can increase under such conditions. The increased pressure can indicate to the hydraulic control system 95 that the distal tip 53 of the needle 51 is within the septum 185. As the distal tip 53 emerges from the septum 185 into the left atrium 190, the pressure within the channel 62 should decrease due to removal or reduction of forces applied between the distal tip 53 and the septum 185. This reduction of pressure in a channel 62 can be measured and recorded by the hydraulic control unit 95. An indication of measured parameters can be displayed to a user. An indication that the distal tip 53 is contacting, puncturing, or traversing the septum 185 can be displayed to the user. An indication that the distal tip 53 has emerged from the septum 185 into the left atrium 190 can be displayed to user. Transition from a collapsed configuration to an extended configuration and/or from an extended configuration to a collapsed configuration can be automated by the hydraulic control unit 95 based on parameters sensed. For example, upon reduction or removal of the return force, the needle 51 can be collapsed by the hydraulic control unit 95.

With the needle 51 spanning the interatrial septum 185, the catheter 11 can follow a path defined by the needle 51 to penetrate the interatrial septum 185 and enter the left atrium 190 through the puncture hole, with a treatment device 31 aligned with the interatrial septum 185, as disclosed herein with respect to FIG. 9B. With the treatment device 31 aligned with the interatrial septum 185, the treatment device 31 can ablate a portion of the interatrial septum 185, as disclosed herein with respect to FIG. 9C.

Various embodiments of the subject technology relate to transseptal devices, systems, and methods suitable to puncture and penetrate the interatrial septum within a heart. One of ordinary skill in the art, however, would understand the subject technology to relate to and include applications to puncture and penetrate other anatomic structures without departing from the general intent or teachings of the present disclosure, including, but not limited to, the portal vein, the outflow hepatic vein, the GI tract, and adjacent structures.

While a preferred embodiment has been set forth in detail with reference to the drawings, those skilled in the art who have reviewed the present disclosure will readily appreciate that other embodiments can be realized within the scope of the subject technology. For example, the subject technology can be implemented with any suitable type of catheter for performing any diagnostic or treatment technique that might be needed in the context of the subject technology. Also, any suitable mechanism for deploying and retracting the needle can be used. Furthermore, human and veterinary applications are contemplated. Moreover, a septum can be broadly understood as a barrier between any two compartments in the body, not restricted to the heart. Therefore, the subject technology should be construed as limited only by the claims appended to any non-provisional application claiming the benefit of the present application, or to any patent issuing therefrom.

The foregoing description is provided to enable a person skilled in the art to practice the various configurations described herein. While the subject technology has been particularly described with reference to the various figures and configurations, it should be understood that these are for illustration purposes only and should not be taken as limiting the scope of the subject technology.

There can be many other ways to implement the subject technology. Various functions and elements described herein can be partitioned differently from those shown without departing from the scope of the subject technology. Various modifications to these configurations will be readily apparent to those skilled in the art, and generic principles defined herein can be applied to other configurations. Thus, many changes and modifications can be made to the subject technology, by one having ordinary skill in the art, without departing from the scope of the subject technology.

It is understood that the specific order or hierarchy of steps in the processes disclosed is an illustration of exemplary approaches. Based upon design preferences, it is understood that the specific order or hierarchy of steps in the processes can be rearranged. Some of the steps can be performed simultaneously. The accompanying method claims present elements of the various steps in a sample order, and are not meant to be limited to the specific order or hierarchy presented.

As used herein, the phrase “at least one of” preceding a series of items, with the terms “and” or “or” to separate any of the items, modifies the list as a whole, rather than each member of the list (i.e., each item). The phrase “at least one of” does not require selection of at least one item; rather, the phrase allows a meaning that includes at least one of any one of the items, and/or at least one of any combination of the items, and/or at least one of each of the items. By way of example, the phrases “at least one of A, B, and C” or “at least one of A, B, or C” each refer to only A, only B, or only C; any combination of A, B, and C; and/or at least one of each of A, B, and C.

Terms such as “top,” “bottom,” “front,” “rear” and the like as used in this disclosure should be understood as referring to an arbitrary frame of reference, rather than to the ordinary gravitational frame of reference. Thus, a top surface, a bottom surface, a front surface, and a rear surface can extend upwardly, downwardly, diagonally, or horizontally in a gravitational frame of reference.

Furthermore, to the extent that the term “include,” “have,” or the like is used in the description or the claims, such term is intended to be inclusive in a manner similar to the term “comprise” as “comprise” is interpreted when employed as a transitional word in a claim.

The word “exemplary” is used herein to mean “serving as an example, instance, or illustration.” Any embodiment described herein as “exemplary” is not necessarily to be construed as preferred or advantageous over other embodiments.

A reference to an element in the singular is not intended to mean “one and only one” unless specifically stated, but rather “one or more.” Pronouns in the masculine (e.g., his) include the feminine and neuter gender (e.g., her and its) and vice versa. The term “some” refers to one or more. Underlined and/or italicized headings and subheadings are used for convenience only, do not limit the subject technology, and are not referred to in connection with the interpretation of the description of the subject technology. All structural and functional equivalents to the elements of the various configurations described throughout this disclosure that are known or later come to be known to those of ordinary skill in the art are expressly incorporated herein by reference and intended to be encompassed by the subject technology. Moreover, nothing disclosed herein is intended to be dedicated to the public regardless of whether such disclosure is explicitly recited in the above description.

While certain aspects and embodiments of the subject technology have been described, these have been presented by way of example only, and are not intended to limit the scope of the subject technology. Indeed, the novel methods and systems described herein can be embodied in a variety of other forms without departing from the spirit thereof. The accompanying claims and their equivalents are intended to cover such forms or modifications as would fall within the scope and spirit of the subject technology.

What is claimed is:

1. A treatment system, comprising:
a catheter having a proximal end, a distal end, a lumen extending from the proximal end to the distal end, and a distal region comprising an ablation treatment device on an outer surface of the distal region;
a needle disposed at least partially within the lumen and having a distal tip advanceable relative to the catheter, wherein the needle comprises a plurality of annular stages having a collapsed configuration and an extended configuration, the annular stages having a greater degree of overlap in the collapsed configuration than in the extended configuration; and
a control unit disposed at the proximal end of the catheter and being operatively connected to the ablation treatment device for ablation of a portion of a septum.

2. The treatment system of claim 1, wherein a distal annular stage has a stopper on an outer surface of the distal annular stage and in contact with an inner surface of a proximal annular stage while in the extended configuration.

3. The treatment system of claim 2, wherein the proximal annular stage has an inner collar extending radially inward from the inner surface of the proximal annular stage, the inner collar having an inner cross-sectional dimension less than an outer cross-sectional dimension of the stopper.

4. The treatment system of claim 2, wherein the distal annular stage has an outer collar extending radially outward from the outer surface of the distal annular stage, the outer collar having an outer cross-sectional dimension greater than an inner cross-sectional dimension of the proximal annular stage.

5. The treatment system of claim 1, wherein the distal tip is disposed on a distalmost one of the annular stages.

6. The treatment system of claim 1, wherein a distalmost one of the annular stages is in fluid communication with a fluid controller.

7. The treatment system of claim 1, wherein the ablation treatment device comprises an anode electrode and a cathode electrode.

8. The treatment system of claim 7, wherein the controller is configured to transmit RF signals to at least one of the anode electrode and the cathode electrode.

9. The treatment system of claim 7, wherein the needle is electrically insulated from the catheter.

10. The treatment system of claim 1, wherein the ablation treatment device comprises a passage for directing a flow of cryogenic fluid to the distal region.

11. A method of treating a patient, comprising:
providing a distal end of a catheter to a septum of a heart of the patient;
advancing a distal tip of a needle from a lumen of the catheter to puncture and cross the septum by transitioning a plurality of annular stages of the needle from a collapsed configuration to an extended configuration, the annular stages having a greater degree of overlap in the collapsed configuration than in the extended configuration;
advancing a distal region of the catheter over the needle to span the septum; and
ablating a portion of the septum with an ablation treatment device on an outer surface of the distal region.

12. The method of claim 11, wherein advancing the distal tip comprises advancing a distal annular stage of the needle, carrying the distal tip, relative to a proximal annular stage of the needle.

13. The method of claim 11, wherein advancing the distal tip comprises
increasing a fluid pressure against an inner surface of the distal tip to an extended configuration pressure;
sensing, by a processor, a return force applied by the septum against the distal tip as the distal tip contacts the septum;
sensing, by a processor, when the return force is reduced as the distal tip extends beyond the septum.

14. The method of claim 13, wherein sensing the return force comprises determining that the fluid pressure exceeds the extended configuration pressure.

15. The method of claim 14, further comprising sensing, by a processor, when the return force is reduced comprises determining that the fluid pressure has returned to the extended configuration pressure.

16. The method of claim 11, wherein providing the distal end of the catheter to the septum of a heart of the patient comprises advancing the catheter to a right atrium of the heart.

17. The method of claim 11, further comprising irrigating at least a portion of the heart with a fluid delivered from the lumen of the catheter while the needle is at least partially disposed in the lumen.

18. A method of treating a patient, comprising:
providing a distal end of a catheter to a septum of a heart of the patient while a distal tip of the needle is within a lumen and at least 4 inches proximally of the distal end of the catheter;
advancing the distal tip of a needle from the lumen of the catheter to puncture and cross the septum;
advancing a distal region of the catheter over the needle to span the septum; and
ablating a portion of the septum with an ablation treatment device on an outer surface of the distal region.

19. The method of claim 18, wherein providing the distal end of the catheter to the septum of a heart of the patient comprises advancing the catheter while the distal tip of the needle is within the lumen at least 4 inches from the distal end of the catheter.

20. The method of claim 18, further comprising irrigating at least a portion of the heart with a fluid delivered from the lumen of the catheter while the needle is at least partially disposed in the lumen.

21. The method of claim 18, wherein ablating the portion of the septum comprises sending an RF signal to electrodes of the ablation treatment device.
